(19) United States
(12) Patent Application Publication
(10) Pub. No.: US 2024/0016496 A1
(43) Pub. Date: Jan. 18, 2024

(54) MEDICAL DEVICE FOR OCCLUDING A LEFT ATRIAL APPENDAGE

(71) Applicant: BOSTON SCIENTIFIC SCIMED, INC., Maple Grove, MN (US)

(72) Inventors: Levi Joel Wolterstorff, Saint Paul, MN (US); Joshua Mark Inouye, Maple Grove, MN (US)

(73) Assignee: BOSTON SCIENTIFIC SCIMED, INC., MAPLE GROVE, MN (US)

(21) Appl. No.: 18/220,415

(22) Filed: Jul. 11, 2023

Related U.S. Application Data
(60) Provisional application No. 63/388,802, filed on Jul. 13, 2022.

(51) Int. Cl. A61B 17/12

(52) U.S. Cl. CPC A61B 17/12122; A61B 17/12159; A61B 2017/1205

(57) ABSTRACT

A medical implant for occluding a left atrial appendage may include an expandable framework configured to shift between a collapsed configuration and an expanded configuration, and an occlusive element secured to the expandable framework. The expandable framework includes a proximal hub and a plurality of interconnected struts extending from the proximal hub, the plurality of interconnected struts defining a main body in the expanded configuration. The expandable framework includes a plurality of elongate anchoring appendages extending from the main body, the plurality of elongate anchoring appendages each including a plurality of anchoring barbs extending therefrom.

### CROSS REFERENCE TO RELATED APPLICATIONS

This application claims the benefit of priority of U.S. Provisional Application No. 63/388,802 filed Jul. 13, 2022, the entire disclosure of which is hereby incorporated by reference.

### TECHNICAL FIELD

The present disclosure pertains to medical devices and systems, and methods for manufacturing and using medical devices and systems. More particularly, the present disclosure pertains to medical implants for occluding a left atrial appendage.

### BACKGROUND

Diseases and/or medical conditions that impact the cardiovascular system are prevalent throughout the world. Traditionally, treatment of the cardiovascular system was often conducted by directly accessing the impacted part of the system. More recently, less invasive therapies have been developed, and have gained wide acceptance among patients and clinicians.

Atrial fibrillation is a common sustained cardiac arrhythmia affecting over 30 million people worldwide, according to some estimates. Atrial fibrillation is the irregular, chaotic beating of the upper chambers of the heart. Electrical impulses discharge so rapidly that the atrial muscle quivers or fibrillates. Episodes of atrial fibrillation may last a few minutes or several days. The most serious consequence of atrial fibrillation is ischemic stroke. It has been estimated that up to 20% of all strokes are related to atrial fibrillation. Most atrial fibrillation patients, regardless of the severity of their symptoms or frequency of episodes, require treatment to reduce the risk of stroke. The left atrial appendage is a small organ attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation are found in the left atrial appendage. As a treatment, medical devices have been developed which are positioned in the left atrial appendage and deployed to close off the ostium of the left atrial appendage. Over time, the exposed surface(s) spanning the ostium of the left atrial appendage becomes covered with tissue (a process called endothelization), effectively removing the left atrial appendage from the circulatory system and reducing or eliminating the number of thrombi which may enter the blood stream from the left atrial appendage.

The disclosure relates to medical implants for occluding the left atrial appendage. Of the known medical devices, systems, and methods, each has certain advantages and disadvantages. There is an ongoing need to provide alternative medical devices and systems, as well as alternative methods for manufacturing and using medical devices and systems.

### SUMMARY

In one example, a medical implant for occluding a left atrial appendage may comprise an expandable framework configured to shift between a collapsed configuration and an expanded configuration, and an occlusive element secured to the expandable framework. The expandable framework may include a proximal hub and a plurality of interconnected struts extending from the proximal hub, the plurality of interconnected struts defining a main body in the expanded configuration. The expandable framework may include a plurality of elongate anchoring appendages extending from the main body, the plurality of elongate anchoring appendages each including a plurality of anchoring barbs extending therefrom.

In addition or alternatively to any example disclosed herein, each elongate anchoring appendage of the plurality of elongate anchoring appendages is fixedly attached to the expandable framework at a first end and extends to a free end opposite the first end.

In addition or alternatively to any example disclosed herein, the plurality of elongate anchoring appendages is integrally formed with the main body as a single monolithic structure.

In addition or alternatively to any example disclosed herein, each elongate anchoring appendage of the plurality of elongate anchoring appendages is devoid of a direct connection to any other elongate anchoring appendage of the plurality of elongate anchoring appendages.

In addition or alternatively to any example disclosed herein, the plurality of elongate anchoring appendages is configured to shift between a first configuration and a second configuration relative to the main body when the expandable framework is in the expanded configuration.

In addition or alternatively to any example disclosed herein, in the first configuration, the plurality of elongate anchoring appendages is disposed within an interior of the main body.

In addition or alternatively to any example disclosed herein, in the second configuration, the plurality of elongate anchoring appendages extends away from the main body.

In addition or alternatively to any example disclosed herein, in the second configuration, the plurality of elongate anchoring appendages extends an axial distance distally from the main body and a radial distance outward from the main body.

In addition or alternatively to any example disclosed herein, the axial distance is greater than the radial distance.

In addition or alternatively to any example disclosed herein, the radial distance is greater than the axial distance.

In addition or alternatively to any example disclosed herein, the plurality of elongate anchoring appendages is self-biased toward the second configuration.

In addition or alternatively to any example disclosed herein, the plurality of elongate anchoring appendages is pulled to the first configuration by at least one tether.

In addition or alternatively to any example disclosed herein, a medical implant for occluding a left atrial appendage may comprise an expandable framework configured to shift between a collapsed configuration and an expanded configuration, and an occlusive element secured to the expandable framework. The expandable framework may include a proximal hub and a plurality of interconnected struts extending from the proximal hub to a distal hub to define a main body in the expanded configuration. The expandable framework may include a plurality of elongate anchoring appendages fixedly attached to and extending from an outer perimeter of the main body, the plurality of elongate anchoring appendages each including a plurality of anchoring barbs extending therefrom.

In addition or alternatively to any example disclosed herein, the plurality of elongate anchoring appendages is configured to extend radially outward of the occlusive element in the expanded configuration.

In addition or alternatively to any example disclosed herein, each of the plurality of elongate anchoring appendages is fixedly attached to the outer perimeter of the main body at a single location.

In addition or alternatively to any example disclosed herein, the plurality of elongate anchoring appendages is disposed proximal of the distal hub in the collapsed configuration.

In addition or alternatively to any example disclosed herein, a medical device system may comprise a catheter, a core wire movably disposed within a lumen of the catheter, and a medical implant for occluding a left atrial appendage releasably connected to a distal portion of the core wire. The medical implant may include an expandable framework configured to shift between a collapsed configuration and an expanded configuration, and an occlusive element secured to the expandable framework. The expandable framework may include a proximal hub and a plurality of interconnected struts extending from the proximal hub, the plurality of interconnected struts defining a main body in the expanded configuration. The expandable framework may include a plurality of elongate anchoring appendages extending from the main body, the plurality of elongate anchoring appendages each including a plurality of anchoring barbs extending therefrom.

In addition or alternatively to any example disclosed herein, the expandable framework is disposed in the collapsed configuration when the medical implant is disposed within the lumen of the catheter and the expandable framework is configured to shift toward the expanded configuration when the medical implant is disposed outside of the lumen of the catheter.

In addition or alternatively to any example disclosed herein, the plurality of elongate anchoring appendages is configured to selectively shift between a first configuration and a second configuration when the medical implant is in the expanded configuration.

In addition or alternatively to any example disclosed herein, the plurality of elongate anchoring appendages is configured to engage a wall of the left atrial appendage in the second configuration to retain the medical implant within the left atrial appendage.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 are side views of an example medical device system;

FIGS. 3-4 are perspective views illustrating selected aspects of a medical implant according to the disclosure;

FIGS. 5-7 are partial cross-sectional views illustrating selected aspects related to the deployment of a medical implant according to the disclosure of one of a plurality of strut groups of the expandable framework in a first configuration;

FIGS. 8-9 are perspective views illustrating selected aspects of a medical implant according to the disclosure; and

FIG. 10 is a partial cross-sectional view illustrating deployment of a medical implant according to the disclosure in the left atrial appendage of a patient.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate exemplary aspects of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. It shall be understood that the discussion(s) herein may apply equally to any and/or all of the components for which there are more than one, unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean the maximum outer dimension, “radial extent” may be understood to mean the maximum radial dimension, “longitudinal extent” may be understood to mean the maximum longitudinal dimension, etc. Each instance of an “extent” may be different (e.g., axial, longitudinal, lateral, radial, circumferential, etc.) and will be apparent to the skilled person from the context of the individual usage. In some instances, an “extent” may be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures illustrate selected components and/or arrangements of medical implants, systems, and methods of manufacturing the same. It should be noted that in any given figure, some features of the medical implants, systems, and methods may not be shown, or may be shown schematically, for simplicity. Additional details regarding some elements may be illustrated in other figures in greater detail. The devices and/or methods disclosed herein may provide a number of desirable features and benefits as described in more detail below.

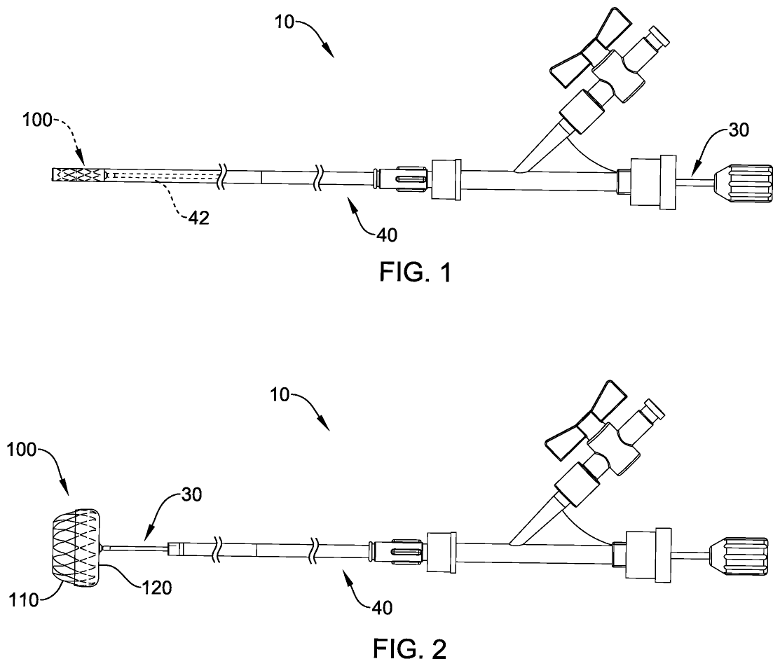

FIGS. 1-2 schematically illustrate selected components and/or arrangements of a medical device system 10. The medical device system 10 may be used to deliver and/or deploy a variety of medical implants (e.g., a cardiovascular medical implant, an occlusive medical implant, etc.) to one or more locations within the anatomy of a patient including but not limited to the heart and/or the vasculature.

The medical device system 10 may include a catheter 40 having a lumen 42 extending from a proximal opening to a distal opening, a core wire 30 movably and/or slidably disposed within the lumen 42, and a medical implant 100 (e.g., a cardiovascular medical implant, an occlusive medical implant, etc.). The medical implant 100 is shown schematically and variations and/or alterations of the example shown are contemplated. The medical implant 100 may be configured to occlude the left atrial appendage of the patient.

The medical implant 100 may include an expandable framework 110 configured to shift between a collapsed configuration (e.g., FIG. 1), wherein the medical implant 100 is disposed within the lumen 42 proximate the distal opening in the collapsed configuration, and an expanded configuration (e.g., FIG. 2), wherein the medical implant 100 and/or the expandable framework 110 is configured to shift radially relative to a central longitudinal axis between the collapsed configuration and the expanded configuration when the medical implant 100 is disposed distal of the distal opening of the lumen 42 and/or the catheter 40, and/or when the medical implant 100 is unconstrained by the catheter 40.

In some embodiments, the medical implant 100 may include an occlusive element 120 disposed on and/or secured to the expandable framework 110. In some embodiments, the occlusive element 120 may be disposed on, along, and/or over an exterior surface of the expandable framework 110. Alternatively, in some embodiments, the occlusive element 120 may encapsulate and/or embed at least a portion of the expandable framework 110 therein.

In some embodiments, the occlusive element 120 may be and/or may include a porous mesh. In some embodiments, the occlusive element 120 and/or the porous mesh may be a woven structure, a fabric structure, a textile structure, and/or a membrane or film having a plurality of apertures formed therein and/or extending therethrough.

In some embodiments, the occlusive element 120 may cover at least 20% of the expandable framework 110 in the expanded configuration. In some embodiments, the occlusive element 120 may cover at least 30% of the expandable framework 110 in the expanded configuration. In some embodiments, the occlusive element 120 may cover at least 40% of the expandable framework 110 in the expanded configuration. In some embodiments, the occlusive element 120 may cover at least 50% of the expandable framework 110 in the expanded configuration. In some embodiments, the occlusive element 120 may cover at least 60% of the expandable framework 110 in the expanded configuration. In some embodiments, the occlusive element 120 may cover at least 70% of the expandable framework 110 in the expanded configuration. Other configurations are also contemplated.

The medical implant 100 may be disposed at and/or releasably connected to a distal portion of the core wire 30. In some embodiments, the medical implant 100 may be releasably connected to the distal end of the core wire 30. The core wire 30 may be slidably and/or rotatably disposed within the lumen 42 of the catheter 40. In some embodiments, a proximal end of the core wire 30 may extend proximally of a proximal end of the catheter 40 and/or the proximal opening of the lumen 42 for manual manipulation by a clinician or practitioner.

Some suitable, but non-limiting, examples of materials for the medical device system the core wire 30, the catheter 40, and/or the medical implant 100, etc. are discussed below. It is contemplated that any and/or all embodiments and/or configurations of the medical implant 100 disclosed herein may be used in accordance with and/or be associated with the medical device system 10 described above.

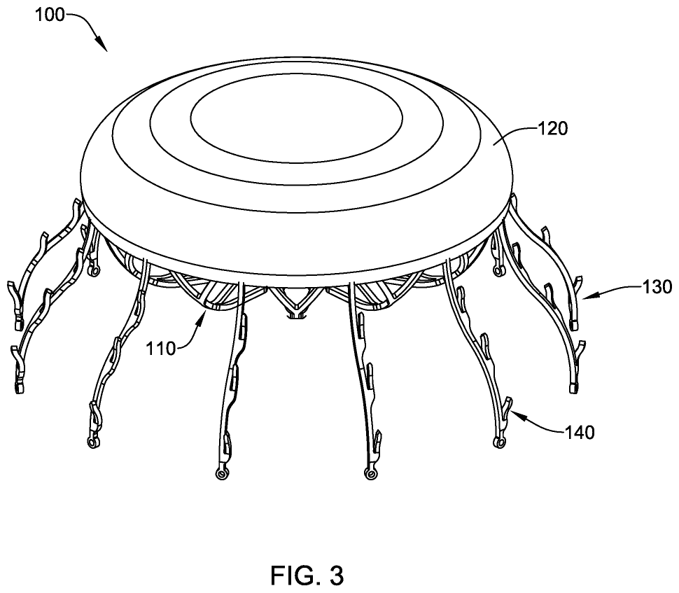

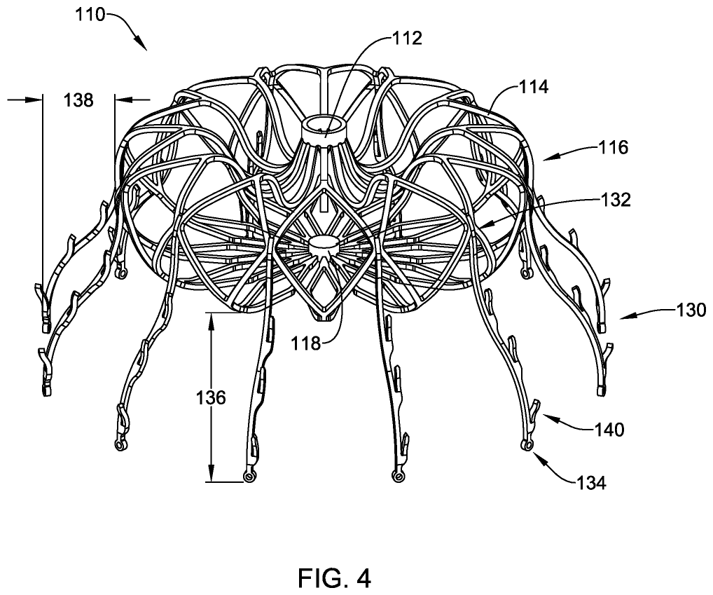

FIGS. 3-4 illustrate selected aspects of the medical implant 100 according to the disclosure. The medical implant 100 includes the expandable framework 110 configured to shift between the collapsed configuration (e.g., FIG. 1) and the expanded configuration (e.g., FIG. 2-4). The expandable framework 110 may include a proximal hub 112 and/or a plurality of interconnected struts 114 extending from the proximal hub 112. In some embodiments, the expandable framework 110 may optionally include a distal hub 118. In at least some embodiments, the plurality of interconnected struts 114 may extend from the proximal hub 112 to the distal hub 118. The expandable framework 110 may have a central longitudinal axis extending from the proximal hub 112 to the distal hub 118 (where present).

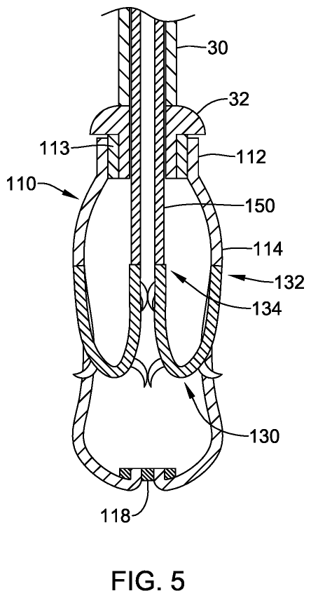

The proximal hub 112 of the expandable framework 110 may be configured to releasably attach or connect to the distal end of the core wire 30. The proximal hub 112 of the expandable framework 110 may include a threaded insert 113 (e.g., FIGS. 5-7) fixedly attached to the expandable framework 110 and/or the proximal hub 112, the threaded insert 113 being configured to engage a threaded member 32 disposed at the distal end of the core wire 30 of the medical device system 10 to releasably connect the medical implant 100 and/or the expandable framework 110 to the distal end of the core wire 30. The expandable framework 110 may be disposed in the collapsed configuration when the medical implant 100 is disposed within the lumen 42 of the catheter 40 of the medical device system 10. The expandable framework 110 may be configured to shift toward the expanded configuration when the medical implant 100 is disposed outside of the lumen 42 of the catheter 40 of the medical device system 10 and/or when the medical implant 100 and/or the expandable framework 110 is unconstrained by the catheter 40.

The plurality of interconnected struts 114 may define a main body 116 of the expandable framework 110 in the expanded configuration. In some embodiments, the plurality of interconnected struts 114 may extend from the proximal hub 112 to the distal hub 118 to define the main body 116 in the expanded configuration. The main body 116 may extend radially outward from the central longitudinal axis. In some embodiments, the main body 116 may have a transverse width measured perpendicular to the central longitudinal axis (e.g., a diameter) that is greater than an axial length measured parallel to the central longitudinal axis in the expanded configuration. Other configurations are also contemplated.

The expandable framework 110 may include a plurality of elongate anchoring appendages 130 extending from the main body 116. In some embodiments, the plurality of elongate anchoring appendages 130 may be fixedly attached to the expandable framework 110 and/or the main body 116 at a first end 132 and extend to a free end 134 opposite the first end 132. In some embodiments, the plurality of elongate anchoring appendages 130 may be fixedly attached to and may extend from an outer perimeter of the main body 116. In some embodiments, each of the plurality of elongate anchoring appendages 130 may be fixedly attached to the outer perimeter of the main body 116 at a single location or at a single attachment point. In at least some embodiments, each elongate anchoring appendage of the plurality of elongate anchoring appendages 130 may be devoid of and/or may be expressly without a direct connection and/or a direct attachment to any other elongate anchoring appendage of the plurality of elongate anchoring appendages 130.

In some embodiments, the plurality of elongate anchoring appendages 130 may each include a plurality of anchoring barbs 140 extending therefrom. In some embodiments, the plurality of anchoring barbs 140 may include two anchoring barbs, three anchoring barbs, four anchoring barbs, etc. Other configurations, including embodiments where each of the plurality of elongate anchoring appendages 130 includes only one anchoring barb, are also contemplated. Alternatively, in some embodiments, the expandable framework 110 and/or the plurality of elongate anchoring appendages 130 may be devoid of any anchoring barbs extending and/or projecting therefrom.

In one non-limiting example, the plurality of anchoring barbs 140 may be shaped in a J-hook configuration when the medical implant 100 and/or the expandable framework 110 is in the expanded configuration. A free end or tip of the J-hook may extend in a proximal direction when the medical implant 100 and/or the expandable framework 110 is in the expanded configuration. In another non-limiting example, the plurality of anchoring barbs 140 may extend proximally from their respective elongate anchoring appendage at an oblique angle to the elongate anchoring appendage when the medical implant 100 and/or the expandable framework 110 is in the expanded configuration. In some embodiments, the plurality of anchoring barbs 140 may be formed separately from the expandable framework 110 and/or the plurality of elongate anchoring appendages 130 and subsequently fixedly attached thereto. In some embodiments, the plurality of anchoring barbs 140 may be integrally formed with the expandable framework 110 and/or the plurality of elongate anchoring appendages 130 as a single monolithic structure.

In some embodiments, the expandable framework 110 may be formed and/or cut from a unitary tubular member. Alternatively, the expandable framework 110 may be formed and/or cut from a flat sheet of material that is later rolled and/or formed into a tubular member. After forming the flat sheet of material into a tubular member, the tubular member may be welded or otherwise fixedly secured into a tubular shape. In some embodiments, forming and/or cutting the expandable framework 110 may be done via laser, waterjet, machining, etc. Other manufacturing methods and/or processes are also contemplated.

In some embodiments, the plurality of elongate anchoring appendages 130 may be integrally formed with the plurality of interconnected struts 114 and/or the main body 116 as a single monolithic structure to form the expandable framework 110 as a whole. In some alternative embodiments, the plurality of elongate anchoring appendages 130 may be formed separately and subsequently fixedly attached the plurality of interconnected struts 114 and/or the main body 116 to form the expandable framework 110 as a whole. In some embodiments, the plurality of elongate anchoring appendages 130 may be fixedly attached to the plurality of interconnected struts 114 and/or the main body 116 by welding, brazing, adhesive bonding, etc. Other configurations are also contemplated. In some embodiments, after forming and/or heat setting the medical implant 100 and/or the expandable framework 110 into the expanded configuration, the occlusive element 120 may be secured to the expandable framework 110.

In some embodiments, one or more of the plurality of elongate anchoring appendages 130 may extend through the occlusive element 120. In some embodiments, at least some of the plurality of anchoring barbs 140 may extend through the occlusive element 120. In some embodiments, the occlusive element 120 may be secured to the expandable framework 110 and/or the plurality of interconnected struts 114 using a plurality of small hooks on the main body 116. In some embodiments, the occlusive element 120 may be secured to the expandable framework 110 using adhesives or other suitable attachment means. In some embodiments, a portion of the expandable framework 110, the plurality of interconnected struts 114, and/or the main body 116 may be embedded within the occlusive element 120. Other configurations are al so contemplated.

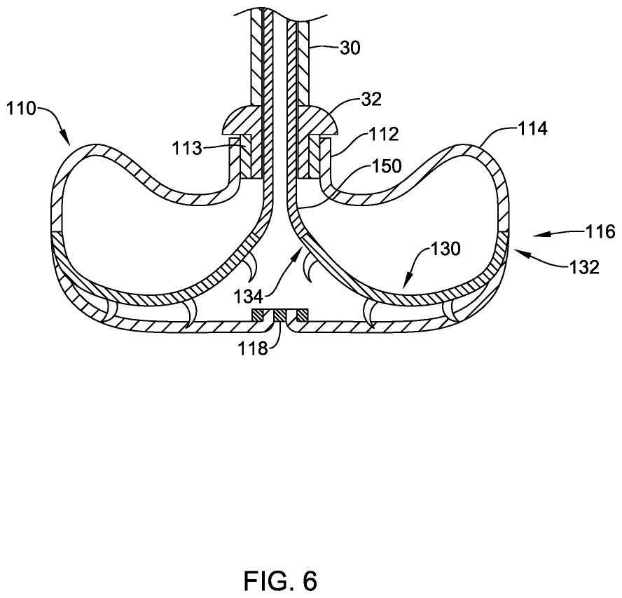

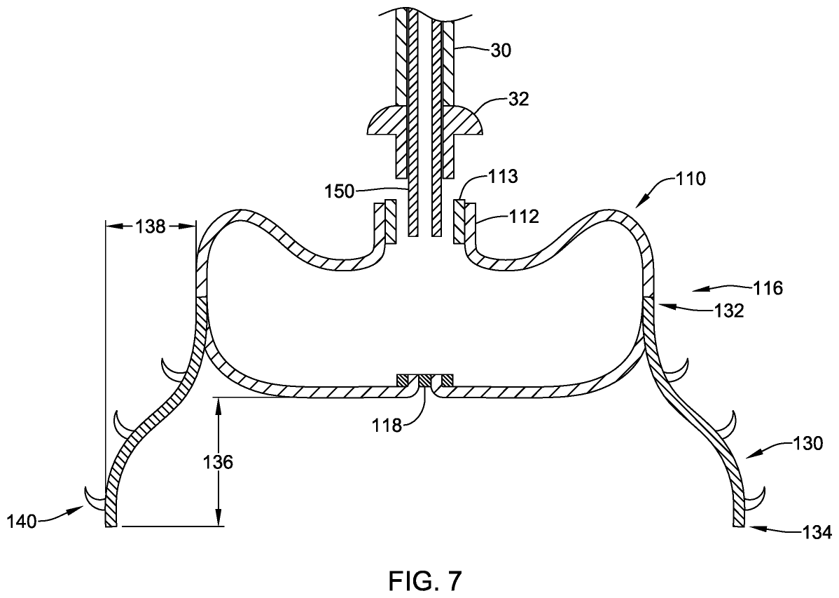

FIGS. 5-7 are partial cross-sectional views illustrating selected aspects related to deploying the medical implant 100. FIG. 5 illustrates the medical implant 100 and/or the expandable framework 110 in the collapsed configuration and coupled to the distal portion of the core wire 30 via the threaded member 32 and the threaded insert 113. In some embodiments, the plurality of elongate anchoring appendages 130 may be disposed proximal of the distal hub 118 (where present) in the collapsed configuration. FIG. 6 illustrates the medical implant 100 and/or the expandable framework 110 in the expanded configuration. As may be seen from FIGS. 6-7, in some embodiments, the plurality of elongate anchoring appendages 130 may be configured to shift between a first configuration (e.g., FIG. 6) and a second configuration (e.g., FIG. 7) relative to the main body 116 when the expandable framework 110 is in the expanded configuration. As shown in FIG. 6, in the first configuration, the plurality of elongate anchoring appendages 130 may be disposed within an interior of the main body 116 and/or radially inward of a hypothetical cylinder defined by the central longitudinal axis and the outer perimeter of the main body 116. As shown in FIG. 7, in the second configuration (and when the expandable framework 110 is in the expanded configuration), the plurality of elongate anchoring appendages 130 may extend away from the main body 116 and/or may extend radially outward of the hypothetical cylinder defined by the central longitudinal axis and the outer perimeter of the main body 116.

As shown in FIGS. 4 and 7, in the second configuration (and when the expandable framework 110 is in the expanded configuration), the plurality of elongate anchoring appendages 130 may extend an axial distance 136 distally from the main body 116 and a radial distance 138 outward from the main body 116, the hypothetical cylinder defined by the central longitudinal axis and the outer perimeter of the main body 116, and/or the occlusive element 120. In some embodiments, in the second configuration (and when the expandable framework 110 is in the expanded configuration), at least some elongate anchoring appendages of the plurality of elongate anchoring appendages 130 may extend an axial distance 136 distally from the main body 116 and a radial distance 138 outward from the main body 116, the hypothetical cylinder defined by the central longitudinal axis and the outer perimeter of the main body 116, and/or the occlusive element 120. In some embodiments, in the second configuration (and when the expandable framework 110 is in the expanded configuration), each elongate anchoring appendage of the plurality of elongate anchoring appendages 130 may extend an axial distance 136 distally from the main body 116 and a radial distance 138 outward from the main body 116, the hypothetical cylinder defined by the central longitudinal axis and the outer perimeter of the main body 116, and/or the occlusive element 120. In at least some embodiments, the axial distance 136 may be greater than the radial distance 138.

In some embodiments, the plurality of elongate anchoring appendages 130 may be self-biased toward the second configuration. In some embodiments, the plurality of elongate anchoring appendages 130 may be pulled into the interior of the main body 116 and/or to the first configuration by at least one tether 150. In some embodiments, tension applied to the free end 134 of the plurality of elongate anchoring appendages 130 using the at least one tether 150 may keep and/or maintain the plurality of elongate anchoring appendages 130 in the first configuration. The at least one tether 150 may extend through the proximal hub 112 into the interior of the main body 116. In some embodiments, the at least one tether 150 may extend within, through, and/or alongside the core wire 30 to the free end 134 of the plurality of elongate anchoring appendages 130.

The at least one tether 150 may be releasably coupled to the free end 134 of the plurality of elongate anchoring appendages 130. In some embodiments, the free end 134 may include an eyelet and the at least one tether 150 may pass through and/or may be detachably connected to the eyelet. It at least some embodiments, the eyelet at the free end 134 may be a closed eyelet having a rounded distalmost end. In some embodiments, the eyelet may have a lateral extent from its respective elongate anchoring appendage and/or a circumferential extent (relative to the central longitudinal axis) that is about 3 times greater than a lateral extent (e.g. width) of its respective elongate anchoring appendage. In some embodiments, the eyelet may have a lateral extent from its respective elongate anchoring appendage and/or a circumferential extent (relative to the central longitudinal axis) that is about 5 times greater than a lateral extent (e.g. width) of its respective elongate anchoring appendage. In some embodiments, the eyelet may have a lateral extent from its respective elongate anchoring appendage and/or a circumferential extent (relative to the central longitudinal axis) that is about 7 times greater than a lateral extent (e.g. width) of its respective elongate anchoring appendage. In some embodiments, the eyelet may have a lateral extent from its respective elongate anchoring appendage and/or a circumferential extent (relative to the central longitudinal axis) that is about times greater than a lateral extent (e.g. width) of its respective elongate anchoring appendage. In some embodiments, the eyelet may have a lateral extent from its respective elongate anchoring appendage and/or a circumferential extent (relative to the central longitudinal axis) that is about 15 times greater than a lateral extent (e.g. width) of its respective elongate anchoring appendage. In some embodiments, the eyelet may have a lateral extent from its respective elongate anchoring appendage and/or a circumferential extent (relative to the central longitudinal axis) that is about 20 times greater than a lateral extent (e.g. width) of its respective elongate anchoring appendage. Other configurations are also contemplated. In at least some embodiments, the eyelet having a lateral or circumferential extent greater than the lateral extent of its respective elongate anchoring appendage may make the plurality of elongate anchoring appendages 130 more atraumatic with respect to tissue (e.g., a wall of the left atrial appendage) at the treatment site.

In some embodiments, each elongate anchoring appendage of the plurality of elongate anchoring appendages 130 may have one tether of the at least one tether 150 associated therewith. For example, and without limitation, a medical implant 100 having twelve elongate anchoring appendages may have at least one tether 150 associated therewith. The quantity twelve is chosen merely as an example, and other quantities (e.g., two, three, four, six, eight, ten, fifteen, twenty, etc.) are also contemplated. In some embodiments, the medical implant 100 having twelve elongate anchoring appendages may include twelve individual tethers—one coupled to each elongate anchoring appendage of the plurality of elongate anchoring appendages 130. In some embodiments, the medical implant 100 having twelve elongate anchoring appendages (and/or the at least one tether 150 associated therewith) may include one core tether extending through the proximal hub 112 and twelve distal tethers, wherein one distal tether extends from the one core tether to each elongate anchoring appendage of the plurality of elongate anchoring appendages 130. Other configurations are also contemplated.

In some embodiments, the plurality of elongate anchoring appendages 130 may be configured to selectively shift between the first configuration and the second configuration when the medical implant 100 and/or the expandable framework 110 is in the expanded configuration. In some embodiments, the plurality of elongate anchoring appendages 130 may be bistable such that the plurality of elongate anchoring appendages 130 may be stable and/or may be self-biased to be in the first configuration and/or the second configuration.

In some alternative embodiments, an external force may be required to shift the plurality of elongate anchoring appendages 130 from the first configuration toward the second configuration, or vice versa. In some embodiments, the plurality of elongate anchoring appendages 130 may be formed from a shape memory material that responds to an external stimulus, such as temperature for example. Other configurations are also contemplated.

As shown in FIGS. 5-6, the plurality of elongate anchoring appendages 130 and/or the plurality of anchoring barbs 140 may be disposed within the interior of the main body 116 of the expandable framework 110 and/or may be spaced away from tissue at a treatment site (e.g., a wall of the left atrial appendage) in the first configuration. This may be beneficial when placing the medical implant 100 because the medical implant 100 may be repositioned, if necessary, while minimizing injury and/or damage to the tissue (e.g., the wall of the left atrial appendage) prior to anchoring and release from the core wire 30. In some embodiments, the plurality of elongate anchoring appendages 130 may be self-biased toward the second configuration, and the plurality of elongate anchoring appendages 130 may be held or retained in the first configuration by tension applied to the at least one tether 150 during deployment of the medical implant 100 and/or as the expandable framework 110 is shifted from the collapsed configuration to the expanded configuration.

Once the practitioner is satisfied with placement with the medical implant 100, which may be verified using a suitable imaging means, the plurality of elongate anchoring appendages 130 may be released from the first configuration and/or may be selectively shifted toward the second configuration, wherein the plurality of elongate anchoring appendages 130 extends away from the expandable framework 110 and/or the main body 116 to engage with the tissue (e.g., the wall of the left atrial appendage) to anchor the medical implant 100 in place. If desired, the at least one tether 150 may be used to pull the plurality of elongate anchoring appendages 130 back toward the first configuration if recapture and/or repositioning of the medical implant 100 is needed.

After deploying the plurality of elongate anchoring appendages 130 and anchoring the medical implant 100 in place, the at least one tether 150 may be released and/or disconnected from the free end 134 of the plurality of elongate anchoring appendages 130, as shown in FIG. 7. In some embodiments, releasing the at least one tether 150 may include cutting the at least one tether 150, easing or releasing tension on the at least one tether 150, releasing one end of the at least one tether 150 as the at least one tether 150 is looped through the eyelet at the free end 134 such that the at least one tether 150 may be pulled through the medical implant 100, etc. Other configurations are also contemplated. Finally, the core wire 30 and/or the threaded member 32 may be disconnected from the medical implant 100, the proximal hub 112, and/or the threaded insert 113, as also shown in FIG. 7.

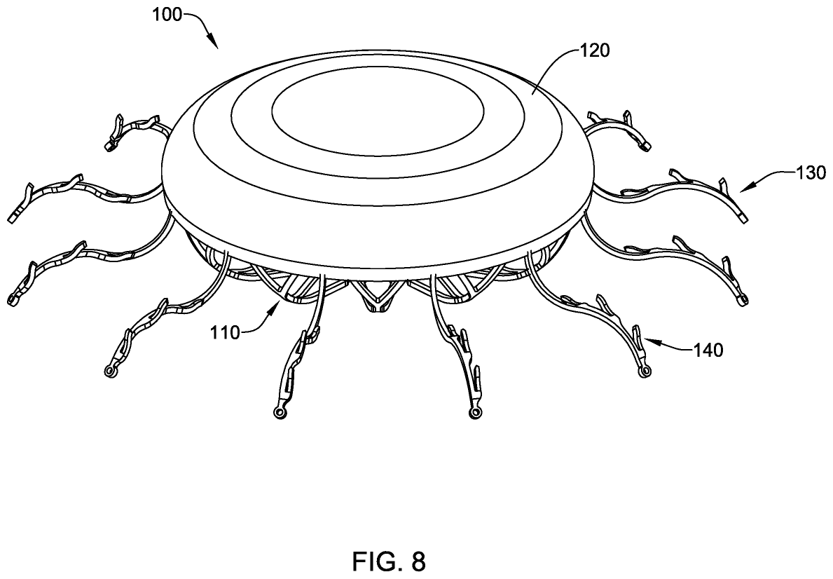

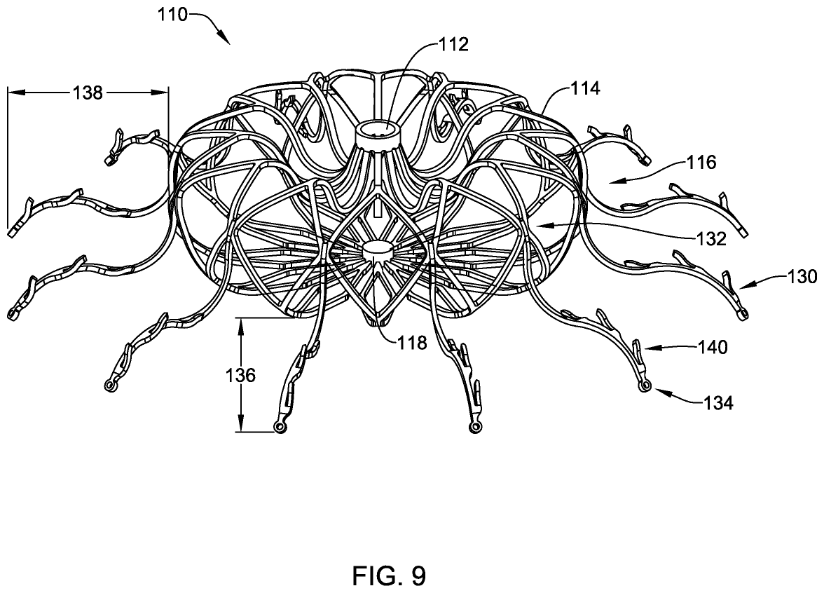

FIGS. 8-9 illustrate selected aspects of an alternative configuration of the medical implant 100 of FIGS. 3-4, wherein the plurality of elongate anchoring appendages 130 is shown in an alternative arrangement of the second configuration. As shown in FIG. 9, and similar to the configuration described above with respect to FIGS. 4 and 7, in the second configuration (and when the expandable framework 110 is in the expanded configuration), the plurality of elongate anchoring appendages 130 may extend an axial distance 136 distally from the main body 116 and a radial distance 138 outward from the main body 116, the hypothetical cylinder defined by the central longitudinal axis and the outer perimeter of the main body 116, and/or the occlusive element 120. In some embodiments, in the second configuration (and when the expandable framework 110 is in the expanded configuration), at least some elongate anchoring appendages of the plurality of elongate anchoring appendages 130 may extend an axial distance 136 distally from the main body 116 and a radial distance 138 outward from the main body 116, the hypothetical cylinder defined by the central longitudinal axis and the outer perimeter of the main body 116, and/or the occlusive element 120. In some embodiments, in the second configuration (and when the expandable framework 110 is in the expanded configuration), each elongate anchoring appendage of the plurality of elongate anchoring appendages 130 may extend an axial distance 136 distally from the main body 116 and a radial distance 138 outward from the main body 116, the hypothetical cylinder defined by the central longitudinal axis and the outer perimeter of the main body 116, and/or the occlusive element 120.

In at least some embodiments, the radial distance 138 may be greater than the axial distance 136, as shown in FIGS. 8-9 for example. In some embodiments, the radial distance 138 being greater than the axial distance 136 may provide additional radial force against tissue (e.g., the wall of the left atrial appendage) at the treatment site if and/or when the plurality of elongate anchoring appendages 130 is compressed radially toward the main body 116 and/or toward the first configuration by the tissue, thereby further improving anchoring of the medical implant 100 within the left atrial appendage.

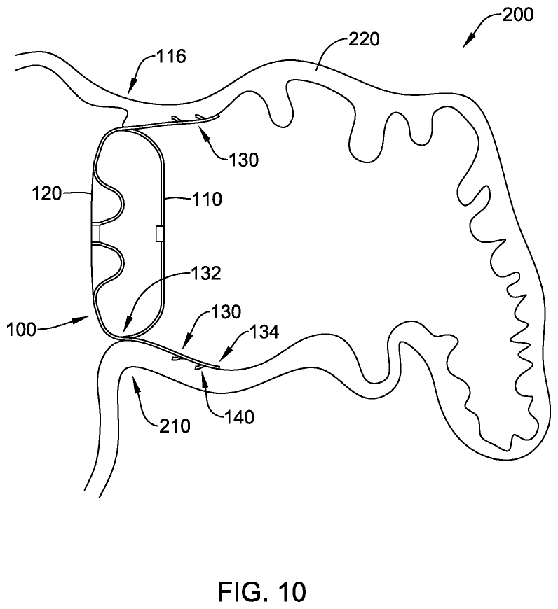

FIG. 10 illustrates selected aspects of a left atrial appendage 200 and the medical implant 100 implanted and/or disposed therein. The left atrial appendage 200 may include an ostium 210 where the left atrial appendage opens into the left atrium of the patient's heart. As seen in FIG. 10, the plurality of elongate anchoring appendages 130 and/or the plurality of anchoring barbs 140 may be configured to engage a wall 220 of the left atrial appendage 200 in the second configuration to retain the medical implant 100 within the left atrial appendage 200. In some patients, the left atrial appendage 200 may be irregularly shaped and/or may include recesses and/or protrusions along the wall 220 thereof. The plurality of elongate anchoring appendages 130 may be sufficiently flexible, while also being biased radially outward, so as to conform to the wall 220 of the left atrial appendage 200 in the second configuration and provide apposition thereto.

The materials that can be used for the various components of the medical implants and/or medical device systems disclosed herein may include those commonly associated with medical devices. For simplicity purposes, the following discussion refers to the system. However, this is not intended to limit the system, devices, and/or methods described herein, as the discussion may be applied to other elements, members, components, or devices disclosed herein, such as, but not limited to, the expandable framework, the plurality of interconnected struts, the plurality of elongate anchoring appendages, the occlusive element, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane (for example, Polyurethane 85A), polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, portions or all of the system and/or components thereof may be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location of the system. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MM) compatibility is imparted into the system. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system may include a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. In some embodiments, the yarns may be made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible system.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-protein and/or anti-bacterial agents (such as 2-methacryroyloxyethyl phosphorylcholine (MPC) and its polymers or copolymers); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical implant for occluding a left atrial appendage, comprising:
an expandable framework configured to shift between a collapsed configuration and an expanded configuration; and
an occlusive element secured to the expandable framework;
wherein the expandable framework includes a proximal hub and a plurality of interconnected struts extending from the proximal hub, the plurality of interconnected struts defining a main body in the expanded configuration;
wherein the expandable framework includes a plurality of elongate anchoring appendages extending from the main body, the plurality of elongate anchoring appendages each including a plurality of anchoring barbs extending therefrom.

2. The medical implant of claim 1, wherein each elongate anchoring appendage of the plurality of elongate anchoring appendages is fixedly attached to the expandable framework at a first end and extends to a free end opposite the first end.

3. The medical implant of claim 2, wherein the plurality of elongate anchoring appendages is integrally formed with the main body as a single monolithic structure.

4. The medical implant of claim 1, wherein each elongate anchoring appendage of the plurality of elongate anchoring appendages is devoid of a direct connection to any other elongate anchoring appendage of the plurality of elongate anchoring appendages.

5. The medical implant of claim 1, wherein the plurality of elongate anchoring appendages is configured to shift between a first configuration and a second configuration relative to the main body when the expandable framework is in the expanded configuration.

6. The medical implant of claim 5, wherein in the first configuration, the plurality of elongate anchoring appendages is disposed within an interior of the main body.

7. The medical implant of claim 5, wherein in the second configuration, the plurality of elongate anchoring appendages extends away from the main body.

8. The medical implant of claim 5, wherein in the second configuration, the plurality of elongate anchoring appendages extends an axial distance distally from the main body and a radial distance outward from the main body.

9. The medical implant of claim 8, wherein the axial distance is greater than the radial distance.

10. The medical implant of claim 8, wherein the radial distance is greater than the axial distance.

11. The medical implant of claim 5, wherein the plurality of elongate anchoring appendages is self-biased toward the second configuration.

12. The medical implant of claim 11, wherein the plurality of elongate anchoring appendages is pulled to the first configuration by at least one tether.

13. A medical implant for occluding a left atrial appendage, comprising:
an expandable framework configured to shift between a collapsed configuration and an expanded configuration; and
an occlusive element secured to the expandable framework;
wherein the expandable framework includes a proximal hub and a plurality of interconnected struts extending from the proximal hub to a distal hub to define a main body in the expanded configuration;
wherein the expandable framework includes a plurality of elongate anchoring appendages fixedly attached to and extending from an outer perimeter of the main body, the plurality of elongate anchoring appendages each including a plurality of anchoring barbs extending therefrom.

14. The medical implant of claim 13, wherein the plurality of elongate anchoring appendages is configured to extend radially outward of the occlusive element in the expanded configuration.

15. The medical implant of claim 13, wherein each of the plurality of elongate anchoring appendages is fixedly attached to the outer perimeter of the main body at a single location.

16. The medical implant of claim 13, wherein the plurality of elongate anchoring appendages is disposed proximal of the distal hub in the collapsed configuration.

17. A medical device system, comprising:
a catheter;
a core wire movably disposed within a lumen of the catheter; and
a medical implant for occluding a left atrial appendage releasably connected to a distal portion of the core wire;
wherein the medical implant includes an expandable framework configured to shift between a collapsed configuration and an expanded configuration, and an occlusive element secured to the expandable framework;
wherein the expandable framework includes a proximal hub and a plurality of interconnected struts extending from the proximal hub, the plurality of interconnected struts defining a main body in the expanded configuration;
wherein the expandable framework includes a plurality of elongate anchoring appendages extending from the main body, the plurality of elongate anchoring appendages each including a plurality of anchoring barbs extending therefrom.

18. The medical device system of claim 17, wherein the expandable framework is disposed in the collapsed configuration when the medical implant is disposed within the lumen of the catheter and the expandable framework is configured to shift toward the expanded configuration when the medical implant is disposed outside of the lumen of the catheter.

19. The medical device system of claim 17, wherein the plurality of elongate anchoring appendages is configured to selectively shift between a first configuration and a second configuration when the medical implant is in the expanded configuration.

20. The medical device system of claim 19, wherein the plurality of elongate anchoring appendages is configured to engage a wall of the left atrial appendage in the second configuration to retain the medical implant within the left atrial appendage.
